(19) United States
(12) Patent Application Publication
(10) Pub. No.: US 2017/0290381 A1
(43) Pub. Date: Oct. 12, 2017

(54) Apparatuses, Methods and Systems for Reinforced Garment Support System

(71) Applicant: SWAT FUEL, INC., Indian Wells, CA (US)

(72) Inventor: Bohdan Theodore Olesnicky, Palm Desert, CA (US)

(21) Appl. No.: 15/358,124

(22) Filed: Nov. 21, 2016

Related U.S. Application Data
(63) Continuation of application No. 12/674,421, filed on Apr. 12, 2011, now abandoned.
(60) Provisional application No. 60/957,142, filed on Aug. 21, 2007.

(51) Int. Cl. A41D 13/05; F41H 1/02; A61F 5/02; A41D 1/04; A41D 3/00

(52) U.S. Cl. CPC A41D 13/0531; A41D 1/04; A41D 3/00; A61F 5/026; F41H 1/02; A41D 2400/22

(57) ABSTRACT

A structural support system for reinforced garments having an axial support member joined to a spinal support member by a quick release mechanism. The axial support member may be incorporated with a reinforced garment belt, while the spinal support member is incorporated with a jacket or vest. The members may be joined by a quick release pin mechanism, so that the jacket may be quickly removed, while the support belt remains in place. The structural support system may be easily adapted based on the needs of a particular user and configured for a variety of applications. For example, the structural support system may be adapted to be incorporated into a reinforced garment or alternately configured to provide external support.

This application claims priority under 35 USC §119(e) of U.S. Provisional Application Ser. No. 60/957,142, filed Aug. 21, 2007, titled “Apparatuses, Methods and Systems For Reinforced Garment Support System”.

### FIELD

The present disclosure generally discusses apparatuses, methods and systems for implementing a ballistic armor support system. More specifically, the present disclosure discusses aspects of a support system that provides support for ballistic armor or any heavy garment, such as lead shielding used in the medical profession.

### BACKGROUND

In certain industries, employees must wear or use equipment that is extremely heavy and/or cumbersome. For example, in the law enforcement industry, the weight of a ballistic armor garment is significant. Furthermore, generally the weight of reinforced garments is often borne primarily by the shoulders and upper body of the user. However, having the full weight of a body armor device borne by the user's upper body leads both to increased levels of user fatigue and the possibility of user injury. Increased user fatigue also may degrade performance and often requires users to take longer, more frequent breaks from work.

### SUMMARY

This disclosure discusses apparatuses, systems and methods that provide a flexible support structure for reinforced garments or certain types of equipment, such as a ballistic armor garment or a bullet-proof vest. The support structure effectively shifts part of the load from reinforced garments/equipment to the lower body and distributes the weight of such reinforced garments between the upper and lower body. An implementation of the support system incorporates a quick release mechanism that facilitates quick removal of the reinforced garment. The structural support system provides significant flexibility and may be adapted for a variety of uses in the law enforcement, security, fire fighting, space exploration industries, the medical industry or any other industry where users may be burdened with heavy, cumbersome garments/equipment.

### BRIEF DESCRIPTION OF THE DRAWINGS

The accompanying appendices and/or drawings illustrate various non-limiting, example, inventive aspects in accordance with the present disclosure:

FIG. 1 illustrates aspects of a structural support system that may be incorporated with reinforced garments according to an implementation of the system.

FIG. 2 illustrates aspects of a quick release mechanism joining a structural spine with an axial support member.

FIG. 3 illustrates an implementation of the structural support system incorporated into a reinforced garment.

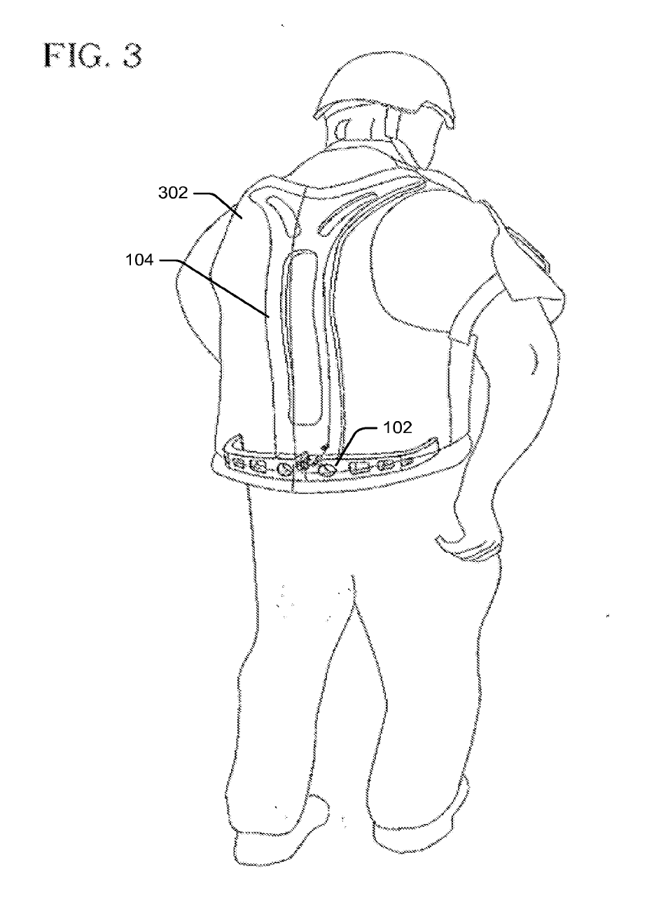

FIG. 4 illustrates aspects of a support spine member of the structural support system according to an implementation of the system.

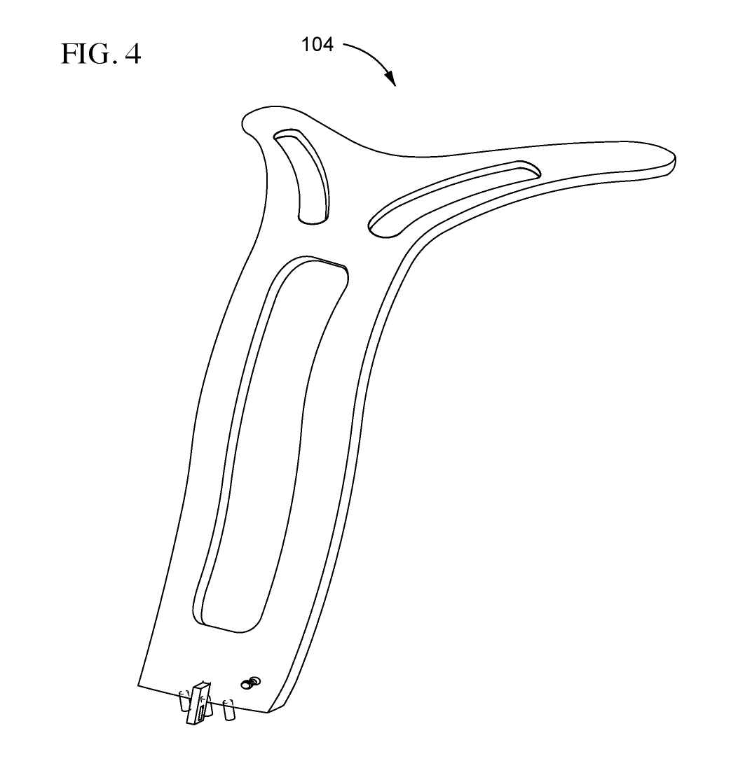

FIG. 5 illustrates aspects of an axial support member of the structural support system according to an implementation of the system.

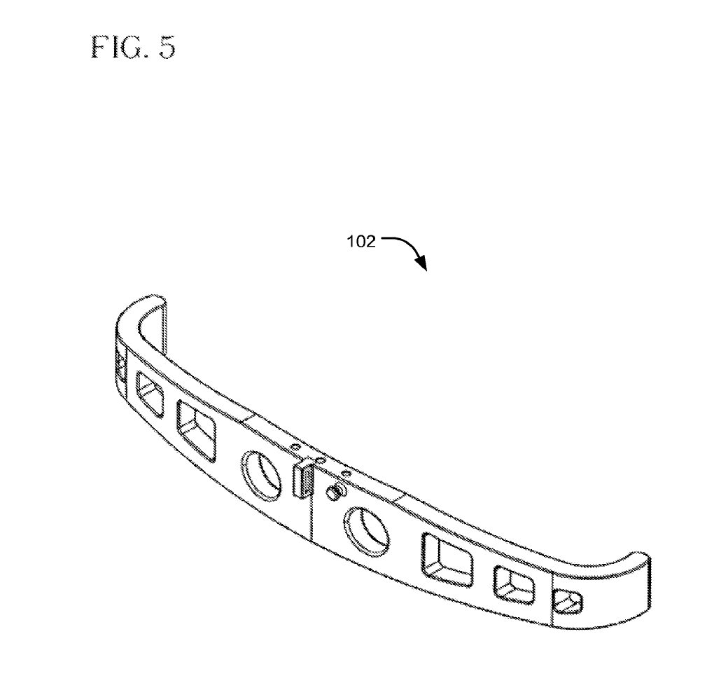

FIG. 6 illustrates aspects of quick release removal of the reinforced garment according to an implementation of the system.

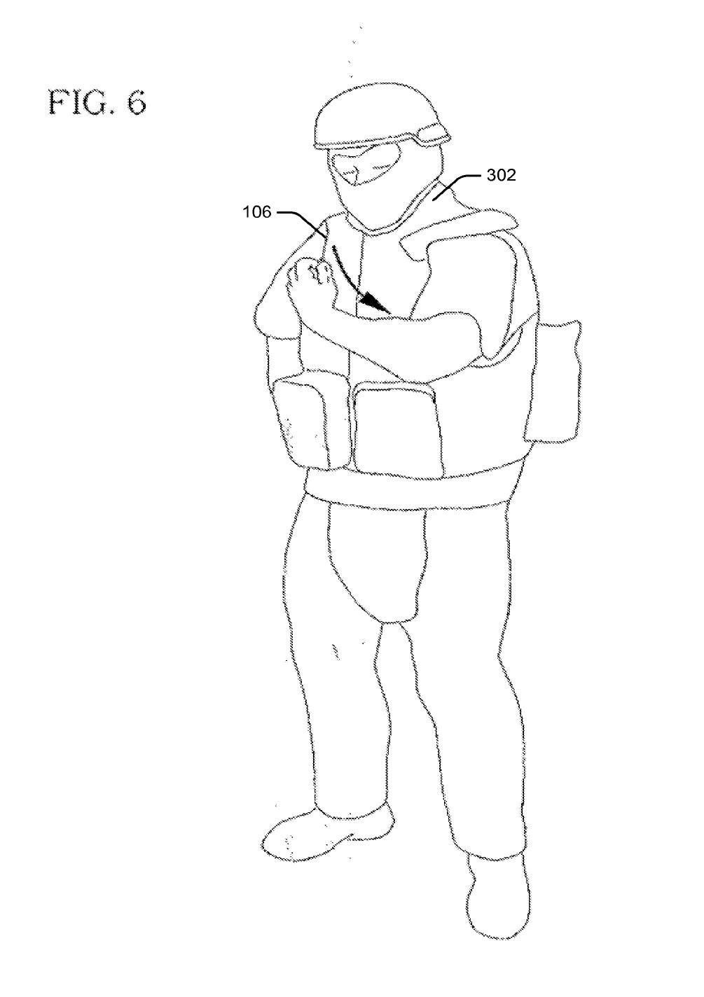

FIG. 7 illustrates additional aspects of quick release removal of the reinforced garment according to an implementation of the system.

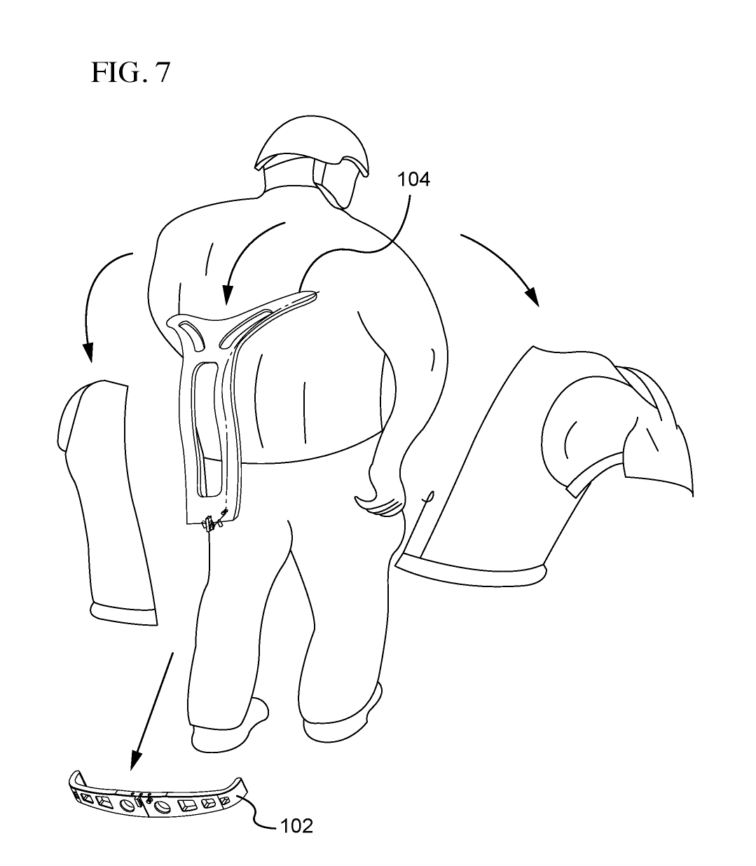

FIG. 8 illustrates aspects of a quick release mechanism of the reinforced garment according to an implementation of the system.

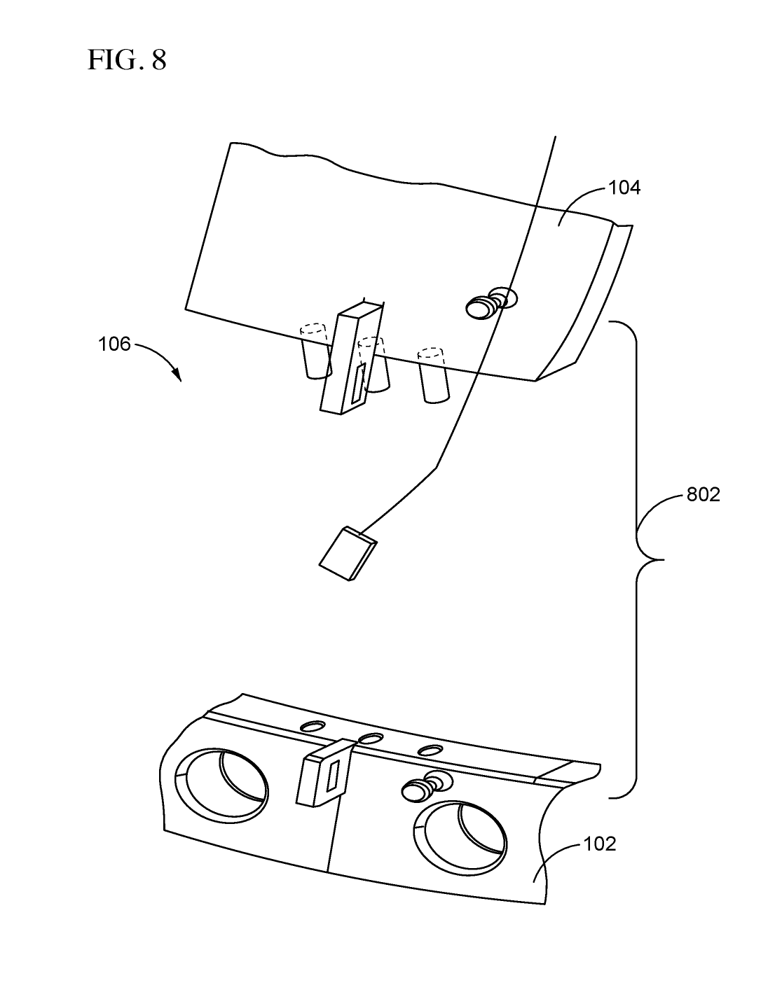

FIG. 9 illustrates aspects of a quick release mechanism of the reinforced garment according to an implementation of the system.

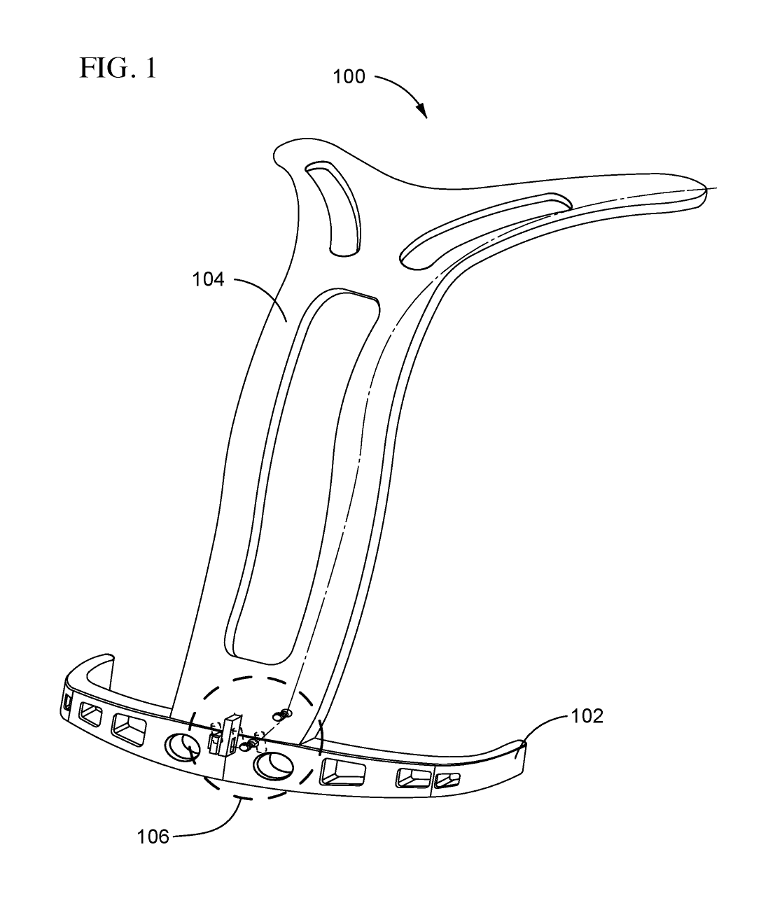

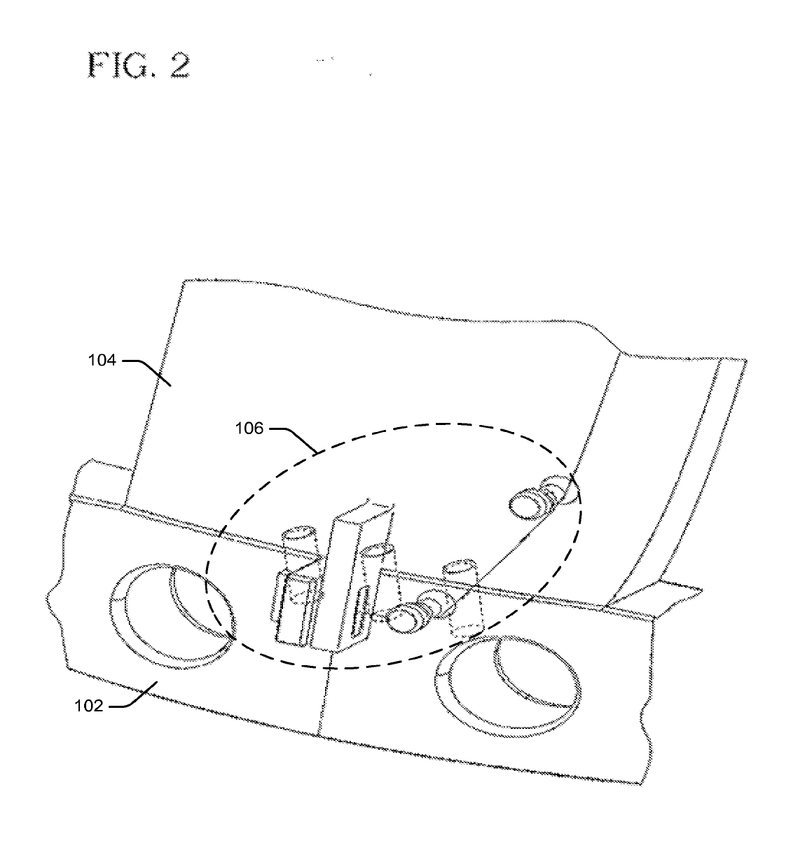

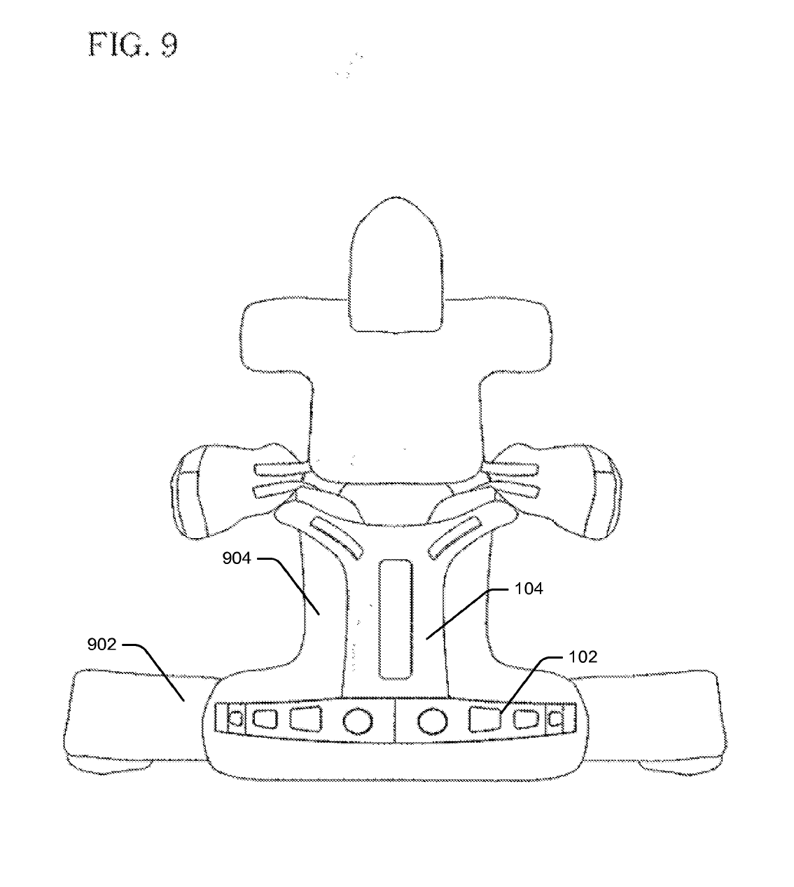

The leading number of each reference number within the drawings indicates the figure in which that reference number is introduced and/or detailed. As such, a detailed discussion of reference number 101 would be found and/or introduced in FIG. 1. Reference number 201 is introduced in FIG. 2, etc.

### DETAILED DESCRIPTION

The present disclosure discusses aspects of apparatuses, methods and systems for implementing a structural supports system for reinforced garments. In an implementation, the structural support system incorporates an axial support member and a structural spine support member. In some implementation, the axial support member and a structural spine support member are fastened with a quick release mechanism. The structural support system provides a great deal of flexibility and may be configured to be incorporated with any number of implementations. However, for the purposes of illustration, the structural support system will be discussed within the context of reinforced garments. Examples of reinforced garments may include ballistic armor used in law enforcement/security industries or lead reinforced garments used in the medical profession, like those used by medical personnel who work with and need protection from certain types of medical equipment (e.g., in radiology, cardiology or a number of other fields). However, it is to be understood that the examples discussed herein are provided for illustrative purposes only and that the structural support system described below may be adapted for any number of implementations based on the needs of a particular user.

An implementation of the structural support system may be configured to be incorporated with a reinforced garment configured as a ballistic armor support system. (BASS). The BASS system is a structural support system for ballistic armor. In various implementations the BASS system may be configured for heavy duty reinforced garments such as those used by police bomb squad users or SWAT team members, a regular configuration for normal, everyday use, and/or a low profile light support configuration for use with reinforced garments that are worn under a user's ordinary clothes or uniform.

The structural support system involves the reduction of the axial load on the human spine by distributing all or part of the load and transferring it to the hips and legs. This is achieved through implementing an accessory support spine member and a pelvic girdle member that consists of two principal parts. The spine member of the apparatus follows the contours of the human spine and may be made of a strong, yet flexible material such as injection molded plastic or carbon fiber. In various implementations, the spine member provides vertical and horizontal support along the spine member, while also allowing for axial twisting and some limited bending, front and back. As discussed above, in various implementations, the thickness of the spinal apparatus can be varied to accommodate different load ranges. The axial member may be configured as a belt portion within the reinforced garment and supports the garment in a way similar to the human pelvic girdle in function, as the axial support member straddles the human pelvis.

The structural support system may be based on joining the spine support member with the axial support member. In an implementation, the axial support member provides an anchor point for the spine portion of the apparatus. The two portions are locked together with a mortise and tenon joint, secured with a quick release pin apparatus. The two portions, together, act as an accessory support spine and pelvis. The structural support system takes the load off of the user's spine, transferring the weight to the human hips and pelvis. Advantageously, the structural support system may be configured to support a reinforced garment. Some implementations may be configured to provide axial twisting, as well as forward and backward bending.

Furthermore, in some implementations of the structural support system, the spine support member may be fastened to the axial support member with a quick release mechanism. As such, the support member may be configured to enable a user to remove the ballistic armor portion of the reinforced garment without having to remove the duty belt portion. This allows for the portions to separate easily in a medical emergency to allow access to an injured user. The belt portion also serves as a padded duty belt for LE applications. Despite the quick release mechanism, the two members should be securely fastened together until such time as the decision is made to separate the components. That is, the two portions cannot separate accidentally as the load then can jar the spine. Some implementations may be configured to be used with an integral harness system with rappelling and/or parachuting reinforced garments.

In an implementation, the BASS may be formed in such a way as to accommodate a breathable padding on the surface of the spine and pelvis to allow for evaporative cooling. As such, the BASS facilitates adequate ventilation for evaporative cooling and to prevent unwanted moisture trapping.

As discussed above, the structural support system facilitates a great deal of flexibility with the design. Depending on the needs of a particular user, the structural support system can be used in a variety of alternate embodiments where a garment carries substantial weight, for example, heavy lead shielding in various medical applications. Furthermore, depending on the particular needs of a user and the configuration, the structural support system may be configured to be incorporated into the reinforced garment or configured to conform to the exterior of the reinforced garment, while still providing support.

The entirety of this disclosure (including the Cover Page, Title, Headings, Field, Background, Summary, Brief Description of the Drawings, Detailed Description, Claims, Abstract, Figures, Appendices and otherwise) shows by way of illustration various embodiments in which the claimed inventions may be practiced. The advantages and features of the disclosure are of a representative sample of embodiments only, and are not exhaustive and/or exclusive. They are presented only to assist in understanding and teach the claimed principles. It should be understood that they are not representative of all claimed inventions. As such, certain aspects of the disclosure have not been discussed herein. That alternate embodiments may not have been presented for a specific portion of the invention or that further undescribed alternate embodiments may be available for a portion is not to be considered a disclaimer of those alternate embodiments. It will be appreciated that many of those undescribed embodiments incorporate the same principles of the invention and others are equivalent. Thus, it is to be understood that other embodiments may be utilized and functional, structural and/or topological modifications may be made without departing from the scope and/or spirit of the disclosure. As such, all examples and/or embodiments are deemed to be non-limiting throughout this disclosure. Also, no inference should be drawn regarding those embodiments discussed herein relative to those not discussed herein other than it is as such for purposes of reducing space and repetition. For instance, it is to be understood that the functional, physical and/or topological structure of any combination of any structural components, as described in the figures and/or throughout are not limited to a particular implementation and/or arrangement, but rather, any disclosed configuration is exemplary and all equivalents, are contemplated by the disclosure. Some of these features may be mutually contradictory, in that they cannot be simultaneously present in a single embodiment. Similarly, some features are applicable to one aspect of the invention, and inapplicable to others. In addition, the disclosure includes other inventions not presently claimed. Applicant reserves all rights in those presently unclaimed inventions including the right to claim such inventions, file additional applications, continuations, continuations in part, divisions, and/or the like thereof. As such, it should be understood that advantages, embodiments, examples, functional, features, structural, topological, and/or other aspects of the disclosure are not to be considered limitations on the disclosure as defined by the claims or limitations on equivalents to the claims.

1-2. (canceled)

3. A reinforced garment structural support system comprising:
an axial support member and a spinal support member configured to transfer the load of a reinforced garment to a user's lower body,
wherein the axial support member is incorporated into a law enforcement duty belt,
wherein the axial support member and the spinal support member are locked together using a mortise and tenon joint,
wherein the axial support member and the spinal support member are each configured with a mortise;
wherein the garment structural support system incorporates a single quick release mechanism through the mortise and tenon joint, said single quick release mechanism comprising a tenon that is configured to fit within the mortise on the axial support member and the mortise on the spinal support member, such that when the tenon is removed, the quick release mechanism detaches the spinal support member from the law enforcement duty belt in a single action disengagement; and
wherein the spinal support member is contoured to correspond with the natural curve of a human spine.

4. The reinforced garment structural support system of claim 3, wherein the quick release mechanism comprises a quick release pin, and wherein the support system is configured so that the axial support member remains in place after the quick release pin and the spinal support member are removed.

5. The system of claim 3, wherein the reinforced garment is a ballistic armor garment.

6. The system of claim 3, wherein the reinforced garment is a bullet-proof vest.

7. The system of claim 3, wherein the reinforced garment is a lead reinforced garment.

8. The system of claim 3, wherein the spinal support member is comprised of injection molded plastic.

9. The system of claim 3, wherein the spinal support member is comprised of carbon fiber.

10. The system of claim 3, wherein the spinal support member is comprised of a twistable material configured to provide axial twisting, and forward and backward bending.

11. The system of claim 3, wherein the spinal support member is incorporated into a jacket portion within the reinforced garment.

12. The system of claim 3, wherein the spinal support member is incorporated into a vest portion within the reinforced garment.

13. The system of claim 3, wherein the belt portion comprises a padded duty belt.

14. The system of claim 3, wherein the axial support member is shaped to straddle a human pelvis.

15. The system of claim 3, wherein the spinal support member further comprises a breathable padding facilitating evaporative ventilation.

16. The system of claim 15, wherein the duty belt further comprises a breathable padding facilitating evaporative ventilation.

17. The system of claim 3, wherein the axial support member and the spinal support member are shaped to conform to the exterior of the reinforced garment.

18. The system of claim 3, wherein the axial support member is connected at an anchor point to the spinal support member.

19. A reinforced garment structural support system, comprising:
a separable reinforced garment comprising a duty belt and a spinal part;
an axial support member incorporated into the duty belt;
a spinal support member incorporated into the spinal part of the reinforced garment, wherein said spinal support member is contoured to correspond with the natural curve of a human spine; and
a single quick release mechanism joining the axial support member and the spinal support member through a mortise and tenon joint, wherein the axial support member and spinal support member are each configured with a mortise, and wherein the single quick release mechanism is a tenon, and when the tenon is removed, the spinal support member detaches from the axial support member in a single action disengagement.

20. A reinforced garment structural support system, comprising:
an axial support means for transferring the load of a reinforced garment across a user's lower body, the axial support means incorporated into a duty belt; and
a spinal support means for transferring the load of a reinforced garment across a vertical axis of a user's torso joined to the axial support means by a quick release mechanism means for facilitating a single action disengagement of the axial support means from the spinal support means,
wherein the quick release mechanism means comprises a mortise and tenon joint, wherein the axial support means and the spinal support means each comprise a mortise, and the quick release mechanism comprises a tenon configured to fit within the mortise on the axial support means and the mortise on the spinal support means, such that when the tenon is removed, the axial support means and the spinal support means are disengaged; and
wherein the spinal support means is contoured to correspond with the natural curve of a human spine.
